(19) United States
(12) Patent Application Publication
(10) Pub. No.: US 2024/0268665 A1
(43) Pub. Date: Aug. 15, 2024

(54) SYSTEM AND METHOD FOR SIMULATION OF FLUORESCEIN ANGIOGRAMS

(71) Applicant: EMAGIX INC., Halifax (CA)

(72) Inventor: Colyn MUNN, Halifax (CA)

(21) Appl. No.: 18/607,959

(22) Filed: Mar. 18, 2024

Related U.S. Application Data
(63) Continuation of application No. PCT/CA2024/050185, filed on Feb. 14, 2024.
(60) Provisional application No. 63/484,753, filed on Feb. 14, 2023.

(51) Int. Cl. A61B 3/12; G06T 7/00; G16H 30/40; G16H 50/50

(52) U.S. Cl. CPC A61B 3/1241; G06T 7/0016; G16H 30/40; G16H 50/50; G06T 2207/10024; G06T 2207/10064; G06T 2207/20081; G06T 2207/20084; G06T 2207/30041

(57) ABSTRACT

In aspects there is provided a computer implemented method and a system for generating a simulated fluorescein angiogram. The method including: receiving a color fundus image at one or more time points; generating a multi-channel two-dimensional pixel array for each time point, wherein one or more of the channels of the pixel array include pixel values from the color fundus image and a third channel of the pixel array includes the encoded time; generating a simulated fluorescein angiography images for each of the time points using a generative network, the generative network taking the multi-channel two-dimensional pixel array as input, the generative network trained using previously captured color fundus images and associated fluorescein angiograms; and outputting the simulated fluorescein angiography images.

### CROSS-REFERENCE TO RELATED APPLICATIONS

This application is a continuation of International Application No. PCT/CA2024/050185, filed Feb. 14, 2024; which claims the benefit of U.S. Provisional Patent Application 63/484,753 filed Feb. 14, 2023, the contents of which are hereby incorporated by reference.

### BACKGROUND

Diabetes affects over 420 million people worldwide. Diabetic retinopathy is the most frequent microvascular complication of diabetes, with over 30% of patients at risk of partial vision loss, and ˜10% at risk of severe visual impairment. Worldwide, diabetic retinopathy is a major cause of blindness and preventable vision impairment. Diabetic retinopathy is caused by dysfunction of retinal blood vessels, and the three hallmarks of the disease include: (i) the appearance of microaneurysms, small circular outpouchings protruding from retinal capillaries; (ii) leakage of the blood-retinal barrier (BRB); and (iii) retinal ischemia. The disease develops and progresses over several stages, with microaneurysms representing the earliest signs of vascular pathology, often appearing before the disease affects vision. As the disease progresses, the vasculature becomes leaky, allowing an influx of blood constituents into the retinal extracellular space. Vascular leakage can eventually result in the development of edema and vision loss. Importantly, BRB leakage and leaky microaneurysms are the main targets of treatments aimed at limiting vision loss. The appearance of ischemia (as a result of capillary occlusion) tends to represent an advanced stage of the disease, with irreversible damage to retinal neuronal-networks and irreparable loss of function. The most severe stage of the disease is characterized by the appearance of retinal neovascularization, and a transition from non-proliferative to proliferative diabetic retinopathy.

Clinical diagnosis of diabetic retinopathy generally includes fluorescein angiography (FA). FA allows the identification of: (i) microaneurysms, appearing as hyperfluorescent dots in the early phase of the scan; (ii) microaneurysm leakage, appearing as fluorescein extravasation in the late phase; (iii) BRB leakage, appearing as non-vascular tissue with fluorescein accumulation in the latent phase of the scan; and (iv) retinal non-perfusion, appearing as tissue that fluorescein does not reach in the early phase of the scan. Another imaging modality that can be useful for the evaluation of diabetic retinopathy is optical coherence tomography (OCT). OCT can be used to capture vascular anatomy and blood flow, and to identify edema and ischemia. This approach generally has a relatively small scan-area. Moreover, this approach does not typically identify mild vascular leakage or mild edema. While microaneurysms can be detected in images acquired using FA, OCT, or color fundoscopy, FA is generally known to outperform the other two approaches in microaneurysm visualization.

FA interpretation has historically been the realm of the retinal specialist. As such, patients are generally referred to ophthalmology by primary care physicians or opticians on the basis of patient-reported history of visual issues or poor performance on visual acuity tests. Diagnosis of diabetic retinopathy is not generally made at first patient contact with the health care system. Many patients with incipient diabetic retinopathy, who might potentially benefit from earlier diagnostic assessment via FA, are currently not being referred due to health system backlogs or because the length and invasiveness of the procedure is deemed not to be warranted (risk/benefit ratio).

By way of example and not an exhaustive list of use cases, FA can be used in the assessment of retinal vein occlusion (RVO), age-related macular degeneration (AMD), hypertensive retinopathy, other retinal conditions, neurovascular conditions, cardiovascular conditions, and other direct and indirect vascular indications. In cases of RVO, FA allows the identification and characterization of venous blockages, revealing delayed venous filling, areas of capillary non-perfusion, and the development of collateral vessels. In AMD, FA is used to detect choroidal neovascularization lesions, and determining their location, size, and activity. Retina specialists use FA to determine the appropriate intervention for preserving vision in patients with diabetic retinopathy, RVO, and AMD. Interventions may include anti-vascular endothelial growth factor (anti-VEGF) therapy and/or laser photocoagulation therapy. Addressing the limitations of FA, particularly its length and invasiveness, will enhance its accessibility for patients with diabetic retinopathy, RVO, and AMD, enabling timelier interventions and better vision outcomes. SUMMARY

Disclosed herein are methods and systems for simulating fluorescein angiograms. Disclosed herein also are methods and systems of training such methods and systems for simulating fluorescein angiograms.

In general, in an aspect, a method of simulating fluorescein angiograms is provided. The method takes as input a fundus image and a set of fluorescein angiography image time points to be simulated. The method uses an AI model that was previously trained on fundus images and their associated fluorescein angiograms to generate a simulated fluorescein angiography image for each of the time points.

In an aspect, a method of simulating fluorescein angiograms is provided. The method begins by receiving a fundus image and a pre-selected list of desired time points. For each of the desired time points in the pre-selected list, the fundus image is then provided as input to an encoder module that outputs to a conversion module, which conversion module then outputs to a decoder module; where the conversion module has input/output connections to a memory module and the encoder module also has skip connections to output directly to the decoder module. Output of the decoder module for each of the desired time points constitutes a predicted fluorescein angiography image at that time point for the provided fundus image.

Implementations of the aforementioned methods may include one or more of the following: The fundus image is a color fundus image. The AI model is a generator network. The encoder module includes one or more sequentially connected encoder blocks. Each encoder block has a convolution layer and an activation layer. The filter size of the convolution layers are 4×4 pixels. The convolution layers use a stride of 2 pixels. There are at least three sequentially connected encoder blocks of increasing convolution filter counts. The number of filters in the sequence increases by powers of two. The number of filters can be 64, 128, 256. The conversion module has a sequential connection of an initial convolution layer, an initial activation layer, and one or more residual blocks. Each residual block has a first convolution layer, a first activation layer, a second convolution layer, a second activation layer, and a skip connection to jump from the front of the block directly to the input of the second activation layer. There are 3, 4, 5, 6, 7, 8, 9, or 10 residual blocks in the conversion module. The decoder module has as many decoder blocks as there are encoder blocks in the encoder module, followed by a convolution and activation layer which outputs the predicted fluorescein angiography image for that time point. Each decoder block includes a transpose convolution layer and activation layer. Each decoder block includes an upsampling layer, convolution layer, and an activation layer. Decoder modules can include multiple architectures of decoder block. There are at least three sequentially connected decoder blocks of decreasing transpose convolution and convolution filter counts. The number of filters in the sequence decreases by powers of two. The number of filters can be, for example, 256, 128, 64. The transpose convolution and convolution layers use a stride of 2 pixels. The filter size of the transpose convolution and convolution layers are 4×4 pixels. The upsampling layer has a kernel size of 2 31×2 pixels and uses nearest neighbor interpolation. The final convolution layer has 3 filters of size 3×3 and uses a stride of 1 pixel.

In an aspect, there is provided a method for analyzing simulated fluorescein angiograms. The method includes providing the simulated fluorescein angiogram and corresponding time points from the method given above into a method previously used for analyzing real fluorescein angiograms (termed RETICAD). Implementations may include one of the following: The analysis method is described in U.S. Pat. No. 9,147,246. The analysis method is described in PCT/CA2022/050926.

In another aspect, there is provided a method for training any of the AI models described above or herein, the method comprising supplying previously captured fundus images and associated fluorescein angiograms as input to an untrained AI model; performing image training comprising building an image discriminator network, wherein the image discriminator network outputs an array of values delineating regions within the simulated fluorescein angiography image that are determined to be simulated, and wherein a goal of training of the AI model comprises reducing elements within the array of values; and performing functional training comprising building a map discriminator network to compare maps of vascular-function determined from the previously captured associated fluorescein angiograms and maps of vascular-function determined from the simulated fluorescein angiography images. In another aspect, there is provided a system for performing the training method mentioned above, the system comprising a processing unit and memory storage, the processing unit in communication with the memory storage and configured to execute the training method.

In another aspect, there is provided a system for predicting fluorescein angiograms, the system comprising a processing unit and memory storage, the processing unit in communication with the memory storage and configured to execute any of the aforementioned methods.

In a particular aspect, a computer implemented method for generating a simulated fluorescein angiogram is provided, the method comprising: receiving a fundus image at one or more time points; generating a multi-channel two-dimensional pixel array for each time point, wherein one or more of the channels of the pixel array comprise pixel values from the color fundus image and a third channel of the pixel array comprises the encoded time; generating a simulated fluorescein angiography images for each of the time points using a generative network, the generative network taking the multi-channel two-dimensional pixel array as input, the generative network trained using previously captured fundus images and associated fluorescein angiograms; and outputting the simulated fluorescein angiography images.

In a particular case of the method, the fundus image is a color fundus image.

In a particular case of the method, the method further comprising determining a mean intensity for each simulated fluorescein angiography image to determine fluorescence, and outputting the simulated fluorescein angiography image with maximal fluorescence.

In a particular case of the method, there are three channels.

In another case of the method, the encoded time comprises a time point relative to a choroidal phase, with respect to a maximum possible time for a fluorescein angiogram.

In yet another case of the method, the encoded time is calculated using a linear function or a logarithmic function.

In yet another case of the method, the generator network comprises a plurality of encoder blocks to compress each color fundus image to a feature representation.

In yet another case of the method, the generator network further comprises a plurality of residual blocks to convert the feature representation of each color fundus image into a feature representation of the corresponding simulated fluorescein angiography image.

In yet another case of the method, the generator network further comprises a plurality of decoder blocks to convert the feature representation of the simulated fluorescein angiography image into the simulated fluorescein angiography image.

In yet another case of the method, the encoder blocks comprise a convolutional layer followed by an activation layer, the residual blocks comprise a convolutional layer followed by an activation layer, and the decoder blocks comprise a transpose convolutional layer followed by an activation layer or an upsampling layer followed by a convolutional layer and an activation layer.

In yet another case of the method, the residual blocks further comprise a second convolutional layer after the activation layer, wherein the first convolutional layer, the activation layer, and the second convolutional later are concatenated with an input to the respective residual block and passed through a second activation layer.

In yet another case of the method, the generator network is trained with an image discriminator network, the image discriminator network outputs an array of values delineating regions within the simulated fluorescein angiography image that are determined to be simulated, and wherein a goal of training of the generator network comprises reducing elements within the array of values.

In yet another case of the method, the generator network is trained using a combination of image training and functional training, the image training comprising using the image discriminator network to reduce elements within the array of values, the functional training comprising a map discriminator network to compare maps of vascular-function determined from the real fluorescein angiogram and maps of vascular-function determined from the simulated fluorescein angiogram.

In yet another case of the method, the outputs of the image discriminator network and the map discriminator network are combined to determine a binary cross entropy loss, which is minimized during training of the generator network.

In yet another case of the method, the map of vascular function comprises one or more of a retinal perfusion map, a retinal blood flow map, a blood-retinal barrier leakage map, and a leaky and non-leaky microaneurysms map.

In another aspect, there is provided a system for generating a simulated fluorescein angiogram, the system comprising one or more processors and a data memory to execute: receiving a color fundus image at one or more time points; generating a multi-channel two-dimensional pixel array for each time point, wherein one or more of the channels of the pixel array comprise pixel values from the color fundus image and a third channel of the pixel array comprises the encoded time value; generating a simulated fluorescein angiography images for each of the time points using a generative network, the generative network taking the multi-channel two-dimensional pixel array as input, the generative network trained using previously captured color fundus images and associated fluorescein angiograms; and outputting the simulated fluorescein angiography images.

In a particular case of the system, the processors further executing: determining a mean intensity for each simulated fluorescein angiography image to determine fluorescence; and outputting the simulated fluorescein angiography image with maximal fluorescence.

In another case of the system, the encoded time comprises a time point relative to a choroidal phase, with respect to a maximum possible time for a fluorescein angiogram.

In another case of the system, the encoded time is calculated using a linear function or a logarithmic function.

In yet another case of the system, the generator network comprises a plurality of encoder blocks to compress each color fundus image to a feature representation, a plurality of residual blocks to convert the feature representation of each color fundus image into a feature representation of the corresponding simulated fluorescein angiography image, and a plurality of decoder blocks to convert the feature representation of the simulated fluorescein angiography image into the simulated fluorescein angiography image.

In yet another case of the system, the encoder blocks comprise a convolutional layer followed by an activation layer, the residual blocks comprise a convolutional layer followed by an activation layer, and the decoder blocks comprise a transpose convolutional layer followed by an activation layer or an upsampling layer followed by a convolutional layer and an activation layer.

In yet another case of the system, the generator network is trained using a combination of image training and functional training, the image training comprising an image discriminator network, the image discriminator network outputs an array of values delineating regions within the simulated fluorescein angiography image that are determined to be simulated, and wherein a goal of training of the generator network comprises reducing elements within the array of values, and the functional training comprising a map discriminator network to compare maps of vascular-function determined from the real fluorescein angiogram and maps of vascular-function determined from the simulated fluorescein angiogram.

In yet another case of the system, the outputs of the image discriminator network and the map discriminator network are combined to determine a binary cross entropy loss, which is minimized during training of the generator network.

These and other aspects are contemplated and described herein. It will be appreciated that the foregoing summary sets out representative aspects of systems and methods to assist skilled readers in understanding the following detailed description.

### BRIEF DESCRIPTION OF THE DRAWINGS

The features of the invention will become more apparent in the following detailed description in which reference is made to the appended drawings wherein:

FIG. 1 is a schematic diagram of a generator network for simulating fluorescein angiograms;

FIG. 2 illustrates examples of an encoder block, residual block, and decoder blocks, in accordance with the method of FIG. 1;

FIG. 3 illustrates an example of the discriminator network, in accordance with the method of FIG. 1;

FIG. 4 illustrates an example diagram of the generator network, in accordance with the example experiment 1;

FIG. 5 is a flowchart diagram of the generator and discriminator network training process;

FIG. 6 illustrates an example of a color fundus image, a sample of four simulated fluorescein angiography images, and an example of a mean-intensity time curve, in accordance with the example experiments;

FIG. 7 illustrates an example of blood-retinal barrier (BRB) leakage, blood flow, and perfusion calculated from a simulated fluorescein angiogram, in accordance with the example experiments;

FIG. 8 is a plot of the linear function and logarithmic function for calculating the encoded time;

FIG. 9 illustrates an example diagram of the generator network, in accordance with the example experiment 2;

FIG. 10 is a flowchart diagram of the RETICAD-augmented generator and discriminator network training process;

FIG. 11 illustrates examples of blood-retinal barrier (BRB) leakage, blood flow, and perfusion calculated in an eye with non-proliferative diabetic retinopathy and diabetic macular edema from: (a) a real fluorescein angiogram; (b) a fluorescein angiogram simulated by a network trained with conventional generative adversarial network (GAN) training; and (c) a fluorescein angiogram by a network trained with RETICAD-enhanced training, in accordance with the example experiments;

FIG. 12 illustrates examples of blood-retinal barrier (BRB) leakage, blood flow, and perfusion calculated in an eye with age-related macular degeneration from: (a) a real fluorescein angiogram; (b) a fluorescein angiogram simulated by a network trained with conventional GAN training; and (c) a fluorescein angiogram by a network trained with RETICAD-enhanced training, in accordance with the example experiments;

FIG. 13 illustrates examples of blood-retinal barrier (BRB) leakage, blood flow, and perfusion calculated in an eye with retinal vein occlusion from: (a) a real fluorescein angiogram; (b) a fluorescein angiogram simulated by a network trained with conventional GAN training; and (c) a fluorescein angiogram by a network trained with RETICAD-enhanced training, in accordance with the example experiments; and

FIG. 14 illustrates examples of blood-retinal barrier (BRB) leakage, blood flow, and perfusion calculated in an eye with age-related macular degeneration and retinal vein occlusion from: (a) a real fluorescein angiogram; (b) a fluorescein angiogram simulated by a network trained with conventional GAN training; and (c) a fluorescein angiogram by a network trained with RETICAD-enhanced training, in accordance with the example experiments.

FIG. 15 illustrates a computer system.

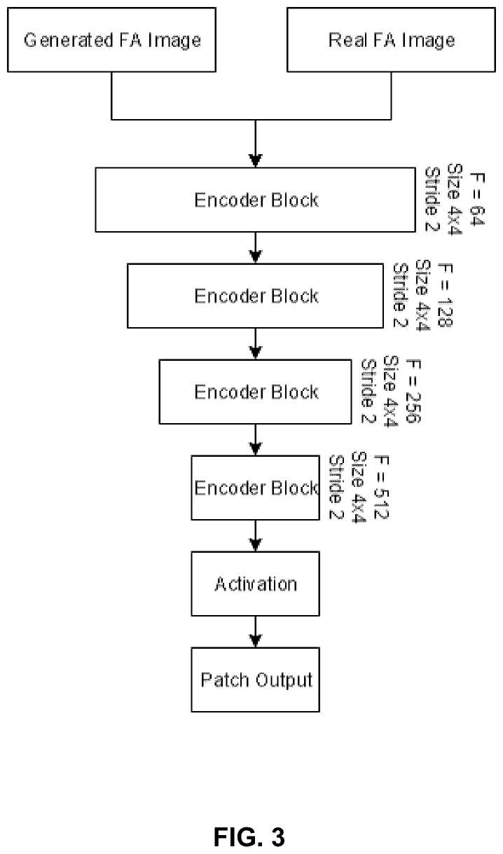

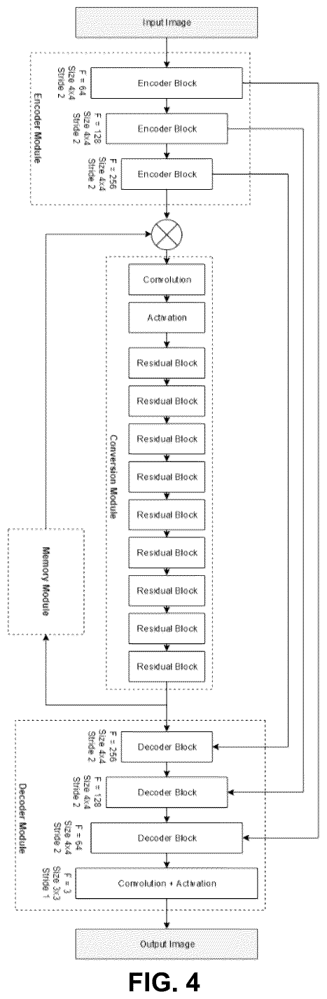

Note in FIGS. 3 and 4, the designation “F=X”, where X is an integer, beside a block represents the number of filters used in the convolution layer (or transpose convolution layer, as applicable) of the block.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

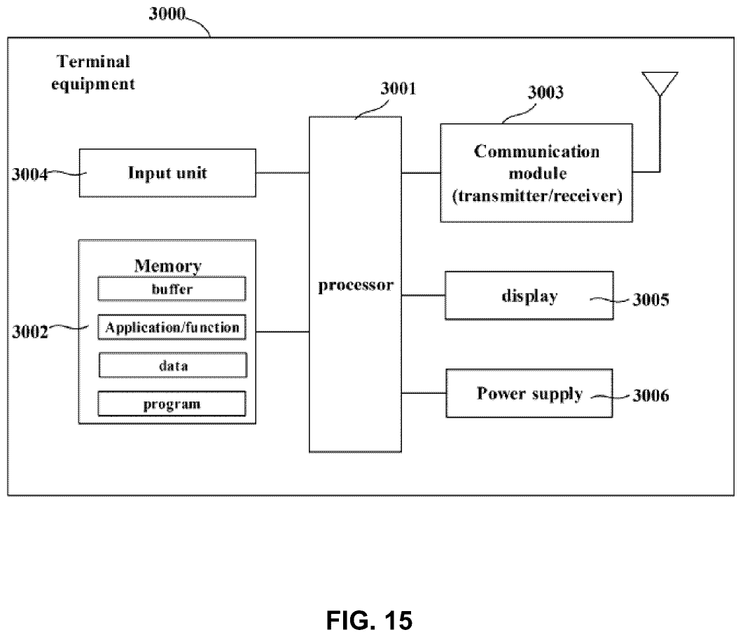

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors. As a non-limiting example, consult FIG. 15 where in some embodiments a computer 3000 has a power supply 3006, a processor 3001, an input unit 3004 for providing input to the processor, memory 3002 for the processor to use as storage for executing applications, functions, instructions, and programs and for accessing buffers and data, a communication module 3003 for transmitting and receiving data e.g., from a network storage device or cloud, and a display 3005 for outputting results.

Fluorescein angiography is an invasive procedure requiring the injection of dye into a patient. Advantageously, embodiments are provided herein that predict fluorescein angiograms using non-invasive fundus imagery, thereby ameliorating the need for fluorescein angiography. In some embodiments, the prediction or simulation of fluorescein angiograms identifies a patient as having insufficient pathology to require the actual procedure, thereby eliminating the need for fluorescein angiography for that patient. In some embodiments, the prediction or simulation of fluorescein angiograms is performed in an environment where fluorescein angiography is not typically practiced. In some embodiments, such environment is an optometrist's office. In some embodiments, such environment is a family physician's office. In some embodiments, such environment is a long-term care facility. In some embodiments, such environment is in a theatre-based military medical centre. In some embodiments, such environment is a primary medical clinic in a developing country. In some embodiments, the prediction or simulation of fluorescein angiograms is performed as part of the decision to refer a patient for fluorescein angiography. In some embodiments, the prediction or simulation of fluorescein angiograms is used in lieu of actual fluorescein angiography, with or without reference to other modalities such as optical coherence tomography angiography.

In some embodiments, the patient has a known or suspected retinopathy and a fluorescein angiography is simulated using a method disclosed herein, followed by a fluorescein angiography analysis method to support the decision for further action. In some embodiments, such method is described in U.S. Pat. No. 9,147,246. In some embodiments, such method is described in PCT/CA2022/050926. In some embodiments, the further action is referral. In some embodiments, the further action is treatment. In some embodiments, the further action is actual fluorescein angiography. In some embodiments, the decision for further action is made by a trained individual such as a retina specialist, an ophthalmologist, an optometrist, an optician, a physician, a medic, a medical photography technology, or by some other individual at least minimally trained to make triage decisions based on a fluorescein angiography analysis method.

To simulate a fluorescein angiogram, methods disclosed herein receive a fundus image and a selection of desired time points as input to an AI model. In some embodiments, the fundus imagery is color fundus imagery. In some embodiments, the desired time points are pre-selected by a user. In some embodiments, the desired time points are selected automatically. In some embodiments, the selected time points are selected so as to provide a representative sampling of the phases of a full-length fluorescein angiogram. In some embodiments, the selected time points are selected so as to provide a representative sampling of an early phase of a fluorescein angiogram. In some embodiments, the selected time points are selected so as to provide a representative sampling of a late phase of a fluorescein angiogram. In some embodiments, the selected time points are selected from a range of time points for which the Al model has been previously trained.

An AI model, suitably trained including as described herein, can be used for the methods and systems of fluorescein angiogram simulation described herein provided the model can transform a fundus image into a set of simulated fluorescein angiogram images at the desired time points. In some embodiments, an AI model takes two or more fundus images from the same patient as input to produce multiple sets of simulated fluorescein angiogram images at the desired time points. 32

In a particular embodiment described below, the AI model is a generator model.

In some embodiments, the color mapping is single channel, (e.g., MONOCHROME1, MONOCHROME2, PALETTE COLOR). In some embodiments, there are three channels, (e.g., RGB, HSV, YBR_FULL, YBR_FULL_422, YBR_PARTIAL_422, YBR_PARTIAL_420, YBR_ICT, YBR_RCT). In some embodiments, there are four channels (e.g., ARGB, CMYK). For each time point, a 2-dimensional array equal in dimensions (height and width) to the color fundus image is created and pixels are assigned values equal to the encoded time. Next, the 2-dimensional arrays are concatenated with copies of the color fundus image. The result is a series of images (the number equal to the total number of desired time points) where color fundus information is 8 contained in the 1st and 2nd channels (e.g., red and green color channels) along with the 9 encoded time in the 3rd channel (e.g., blue color channel), which is then ready to be input into a generator network.

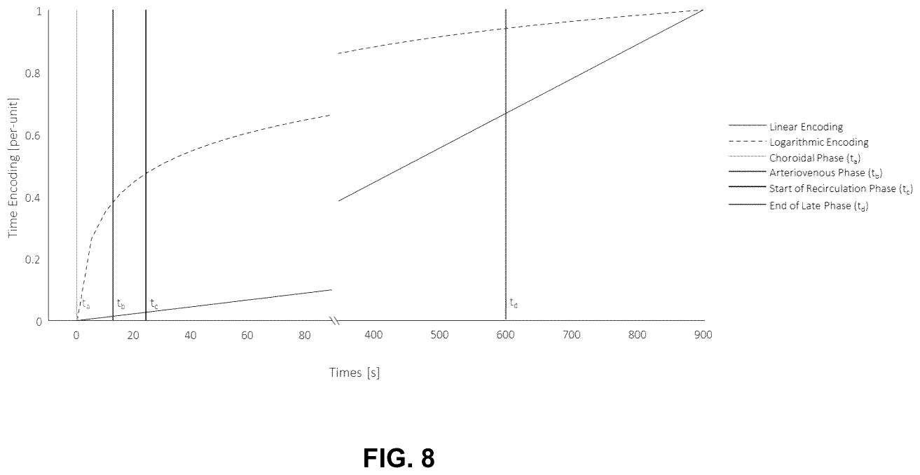

In some embodiments, encoded time is calculated using a linear function (FIG. 8). This process is illustrated by the following mathematical equation:

$${t}_{encode} ( t ) = \frac{t - {t}_{a}}{{t}_{max}}$$

where
t_{encode} is the calculated encoded time,
t is the desired time point in seconds
tₐ is the time of the choroidal phase, for example, 8 seconds post-injection.
tₘₐₓ is the latest possible time of a fluorescein angiogram for example, 900 seconds seconds post-injection.

In some embodiments, encoded time is calculated using a logarithmic function (FIG. 8). This process is illustrated by the following mathematical equation:

$${t}_{encode} ( t ) = \{ \begin{matrix}0 , & t \leq 0 \\ \frac{ln ( t - {t}_{a} + 1 )}{ln ( {t}_{max} + 1 )} , & t > 0\end{matrix}$$

where
t_{encode} is the calculated encoded time,
t is the desired time point in seconds,
tₐ is the time of the choroidal phase, for example, 8 seconds post-injection,
tₘₐₓ is the latest possible time of a fluorescein angiogram, for example, 900 seconds post-injection.

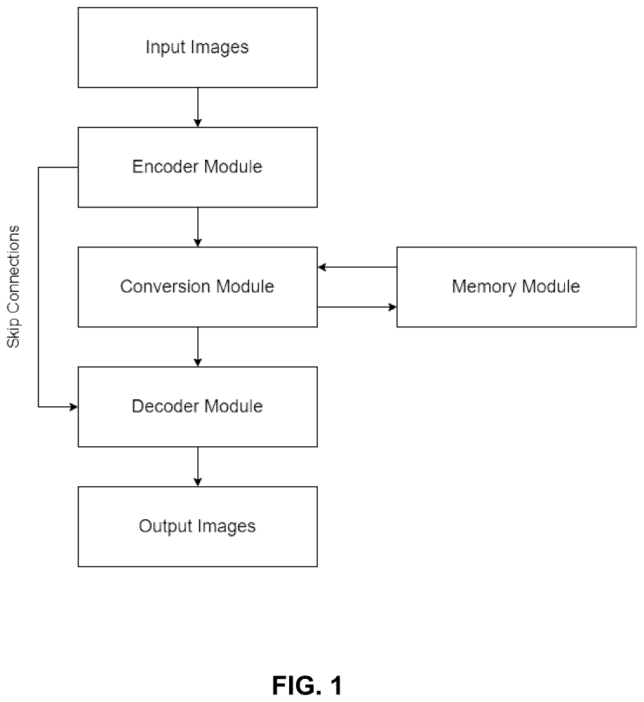

An exemplary generator network, implemented on one or more computer processors and memory, is depicted in FIG. 1. Generator networks used by the methods disclosed herein comprise an encoder module, a conversion module, a decoder module, and a memory module. Such networks operate iteratively to simulate a fluorescein angiogram and can utilize information generated and stored in the memory module from the previous iteration to simulate subsequent FA images until the sequence is of sufficient quality for output. A series of encoder blocks can be used to compress a given input image into a low-

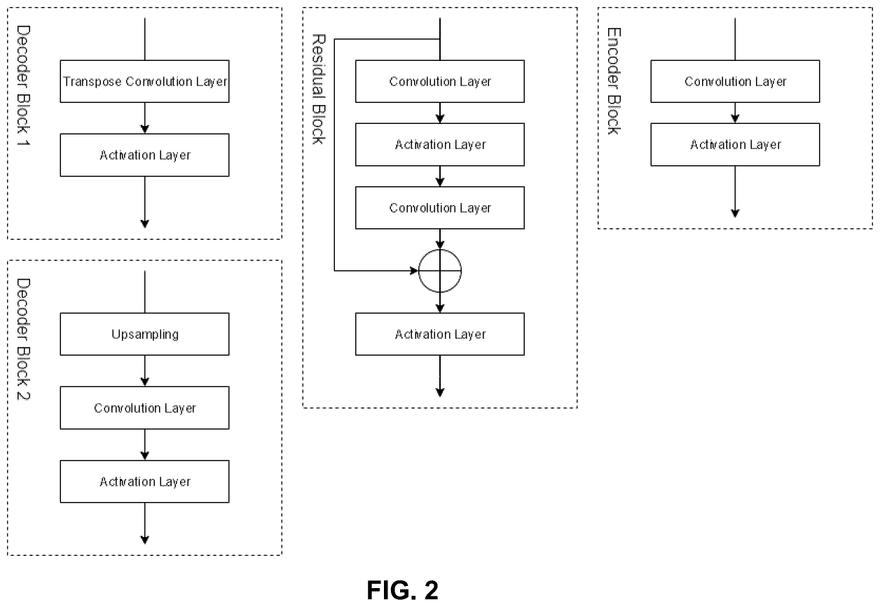

resolution feature representation (see FIG. 2, encoder block). Each encoder block can contain a convolutional layer followed by an activation layer. Convolutional layers have defined kernel size, stride, and may have padding properties. In some embodiments, an encoder block has a kernel size of 4, a stride of 2, and “same” padding. In some embodiments, the activation layer is a leaky rectified linear unit (ReLU) with a negative slope coefficient of 0.2. Additionally, outputs from each encoder block are in some embodiments transferred to the decoder module via skip connections.

A series of residual blocks can be used to convert the feature representation of the input image into a feature representation of its corresponding FA image (see FIG. 2, residual block). In some embodiments, the first block of the conversion module has a convolutional layer followed by an activation layer. In some embodiments, the first block is an encoder block with a convolutional layer having a kernel size of 4, a stride of 2, and “same” padding, and an activation layer using leaky ReLU with a negative slope coefficient of 0.2. Subsequent elements in the conversion model may comprise a residual block with a convolutional layer, followed by an activation layer and a second convolutional layer. The output from these first three layers of the residual block when present can be concatenated with the residual block's input and passed through a further activation layer.

The output from the final residual block in the series can be recorded to the memory module for subsequent image generation. In some embodiments, during a given nth cycle of FA sequence generation via a generator network, the conversion module stores the feature representation of the FA image in the memory module, so that during the n+1th cycle, the conversion module can retrieve the nth cycle's feature representation of the simulated FA image from the memory module and concatenate it with the n+1th cycle's feature representation of the input image received from the encoder module.

In the decoder module, a series of decoder blocks can be used to convert the feature representation of an FA image into a high-resolution FA image (see FIG. 2, decoder block type 1 or 2). Two types of decoder blocks can be used: (1) a block comprising a transpose convolutional layer, followed by an activation layer, or (2) a block comprising an upsampling layer followed by a convolutional layer and an activation layer. The final block in the decoder module can be a convolutional layer (e.g., kernel size of 3, a stride of 1, and “same” padding), 6 followed by an activation layer (e.g., tan h activation function). The output from the final block is a simulated FA image. In some embodiments, a convolutional (or a transpose convolutional as applicable) layer within a decoder block has a kernel size of 4, a stride of 2, and “same” padding, while a final convolutional layer at the end of a decoder block has a kernel size of 3, a stride of 1, and “same” padding. In some embodiments, activation layers within a decoder block use leaky ReLU with a negative slope coefficient of 0.2, while a final activation layer at the end of a decoder block uses a tan h activation function. In some embodiments, an upsampling layer uses nearest neighbor interpolation.

In some embodiments, a discriminator network may be used in the process of training a generator network for use in methods disclosed herein. An exemplary PatchGAN discriminator network architecture is given in FIG. 3. Input of both a simulated FA image and an analogous real FA image to a series of encoder blocks produces a feature representation of a given FA image. In some embodiments, encoder block properties are consistent with those used in the generator network used to output the candidate simulated FA image. In some embodiments, the final block of the network comprises a convolutional layer with kernel size 3, stride 1, and “same” padding followed by an activation layer using a sigmoid function. The discriminator network provides as output a two-dimensional array of values delineating regions as either real or simulated. Each encoder block can consist of a convolutional layer (e.g., kernel size of 4, a stride of 2, and “same” padding), and an activation layer (e.g., leaky ReLU with a negative slope coefficient of 0.2). The final block of the network can consist of a convolutional layer (e.g., kernel size of 3, a stride of 1, and “same” padding), and an activation layer (e.g., sigmoid function). The output of the network is an n×m array of values delineating regions within the candidate simulated FA image that are deemed simulated. In some embodiments, reducing the value of individual elements within the array of values, or of some segments thereof, is a goal of training. Conventional GAN training

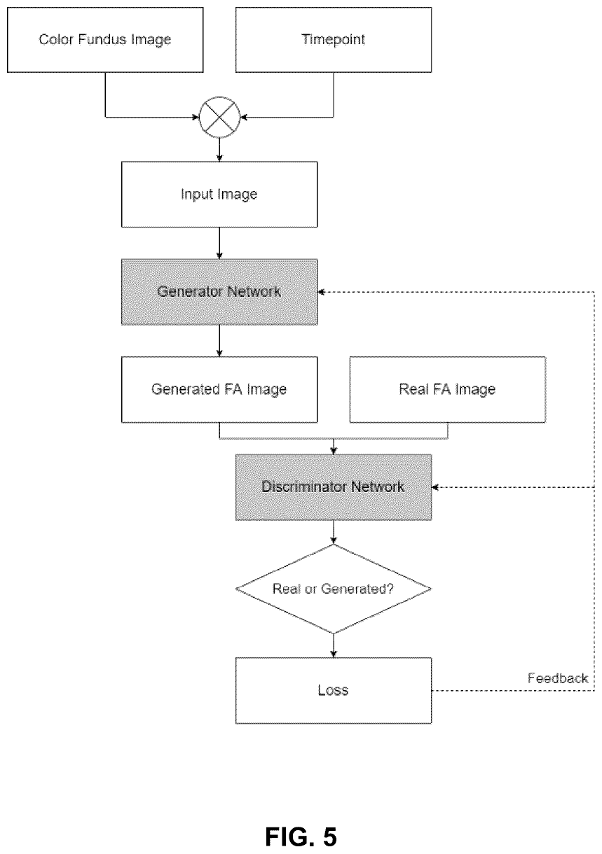

Network training can be conducted using a conventional technique for generative adversarial networks (FIG. 5). Losses for the generator and discriminator networks are calculated using their appropriate loss equation. The generator attempts to minimize the loss for the simulated FA images, while the discriminator network attempts to maximize. In some embodiments, batch normalization can be used to re-center and rescale the data for faster training. In some embodiments, dropout is used with a rate of, e.g., 0.5 in the encoder and residual blocks to prevent overfitting. In some embodiments, weights are randomly initialized using a normal distribution. In some embodiments, the normal distribution assumes a mean of 6 0.0 and a standard deviation of 0.02. In some embodiments, Adam optimization is used. In some embodiments, Adam optimization parameters comprise a learning rate of 0.0002, β₁ of 0.5, β₂ of 0.999, and & of 1e-07. In some embodiments, the loss equation can be defined as the weighted sum of binary cross entropy and mean absolute error (MAE) using weights, for example, of 1 and 100, respectively. In some embodiments, the discriminator's loss function is binary cross entropy with a weight of, e.g., 0.5. In some embodiments, batch normalization can be used to re-center and rescale the data for faster training, optionally using random initialization or Adam optimization with parameters as given above.

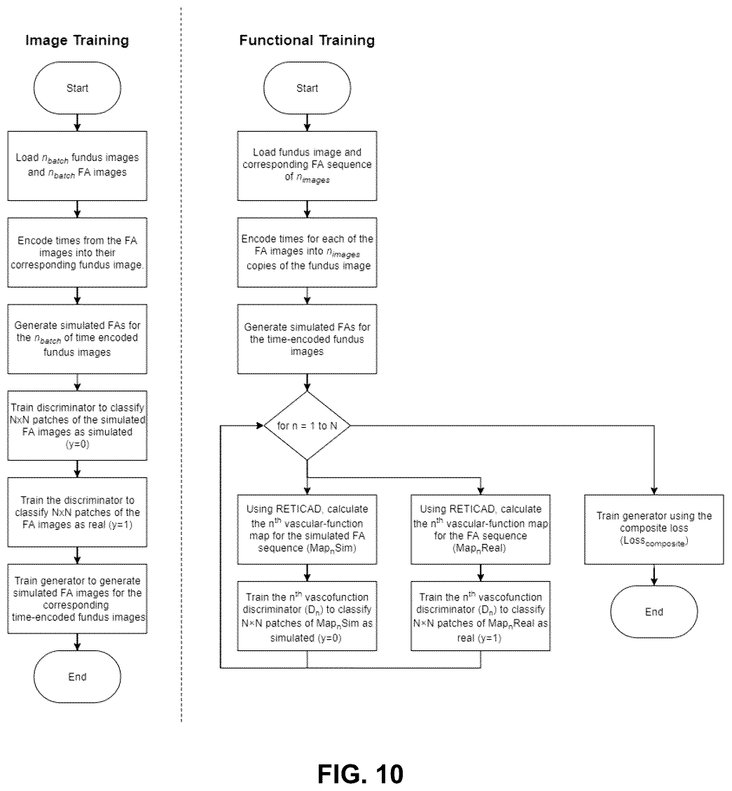

RETICAD-augmented generative adversarial network training RETICAD-augmented GAN training combines two training paradigms: image training and functional training (FIG. 10). During image training, the generator attempts to minimize the loss, while the image discriminator network attempts to maximize. This process is described herein. The functional training involves additional discriminator networks, termed map discriminators. As inputs, these networks receive maps of vascular-function calculated by RETICAD. RETICAD calculates maps of vascular function from a real fluorescein angiogram or simulated fluorescein angiogram. The maps are calculated in a per-pixel analysis of the time series data contained in the real/simulated fluorescein angiograms. The vascular-function maps may include, but do not represent an exhaustive list: (i) retinal perfusion, measured as the rate of fluorescence extra-vascular wash-in during the early phase of the fluorescein angiogram; (ii) retinal blood flow, measured as the rate of fluorescence intra-vascular wash-in during the early phase of the fluorescein angiogram; (iii) BRB leakage, measured as the rate of fluorescence accumulation in extra-vascular tissue across the late phase of the fluorescein angiogram; (iv) leaky and non-leaky microaneurysms, detected as hyperfluorescent dots in the early phase of the fluorescein angiogram, with/without fluorescein extravasation in the late phase, respectively.

For a given instance of functional training (FIG. 10), a color fundus image and its corresponding real sequence of FA images are loaded into memory. For each image of the fluorescein angiogram, a copy of the color fundus image is created, and the time of the fluorescein angiography image is encoded into the copy of the color fundus image. Next, the time-encoded color fundus images are processed by the generator to create a simulated fluorescein angiogram.

The simulated fluorescein angiogram then analyzed using the RETICAD algorithm. The RETICAD algorithm calculates N maps of vascular-function. The N maps are paired with the patient's corresponding color fundus image and used as training data for a set of N map discriminator(s). The N map discriminator(s) can be trained using a conventional discriminator training technique to recognize the N maps as simulated (y=0).

Similarly, the real fluorescein angiogram is then analyzed using the RETICAD algorithm. The RETICAD algorithm calculates N maps of vascular function. The N maps are paired with the patient's corresponding color fundus image and used as training data for the N map discriminator(s). The N map discriminator(s) can be trained using a conventional discriminator training technique to recognize the N maps as real (y=1).

The outputs of each discriminator are used to calculate a binary cross entropy loss (Lossₙ), illustrated by the following mathematical equation:

$${Loss}_{n} = [ {𝔼}_{{Map}_{n} Real} log ( {D}_{n} ( {Map}_{n} Real ) ) + {𝔼}_{{Map}_{n} Sim} log ( 1 - {D}_{n} ( {Map}_{n} Sim ) ) ]$$

where
MapₙReal is a vascular-function map calculated from a real fluorescein angiogram,
MapₙSim is a vascular-function map calculated from a simulated fluorescein angiogram.

During training, the generator attempts to minimize a composite loss (Loss_{composite}), illustrated by the following mathematical equation:

$${Loss}_{composite} = {min}_{G} {max}_{{D}_{n}} {{\sum }^{N}}_{n = 1} {a}_{n} {Loss}_{n}$$

where
N is the number of discriminators,
Lossₙ is the n binary cross entropy loss,
αₙ is the weight of Lossₙ.

In some embodiments, there can be two map discriminators, with composite loss function weights of, e.g., α₁=0.5 and α₂=0.5. The composite loss function can be used in conjunction with optimization methods, e.g., gradient descent, to train the generator.

In some embodiments, batch normalization can be used to re-center and rescale the data for faster training. In some embodiments, the normal distribution assumes a mean of 0.0 and a standard deviation of 0.02. In some embodiments, Adam optimization is used. In some embodiments, Adam optimization parameters comprise a learning rate of 0.0002, β₁ of 0.5, β₂ of 0.999, and ε of 1e−07. In some embodiments, the discriminators' loss function is binary cross entropy with a weight of, e.g., 0.5. In some embodiments, batch normalization can be used to re-center and rescale the data for faster training, optionally using random initialization or Adam optimization with parameters as given above.

Reference is hereby made to an example below utilizing a generator network architecture as generally depicted in FIG. 4, which was trained using methods of training described in FIG. 5 and further below.

### Example 1

Conventional GAN training: simulation of a fluorescein angiogram from a color fundus image followed by analysis of the simulated fluorescein angiogram A network was designed (FIG. 4) and trained using a batch size of 1 across 136 fluorescein angiograms (2,818 images) and corresponding color fundus images (136 images).

The selected model was chosen from the 14th epoch of training and established as the generator network for the generation of simulated fluorescein angiograms. Time-encoding was performed using the linear function as described herein and shown in FIG. 8.

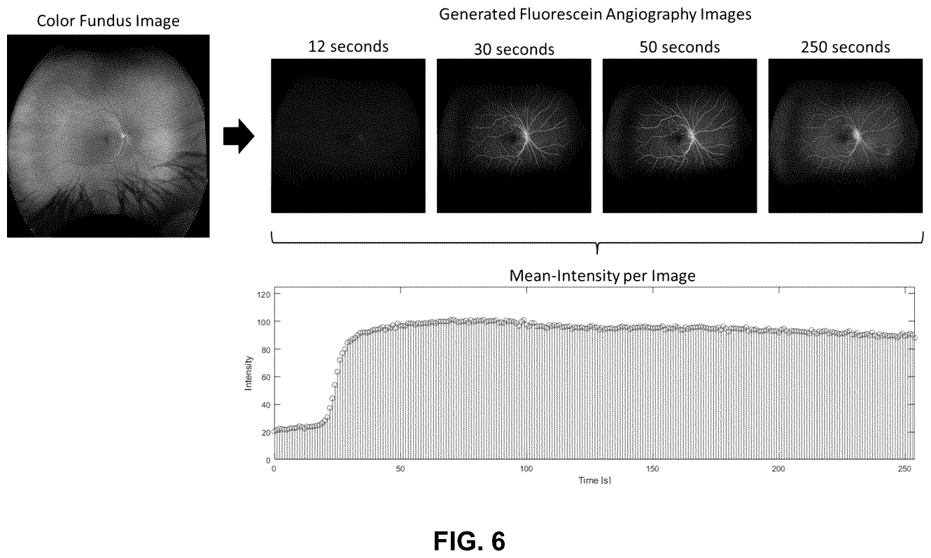

A color fundus image was selected from a test set and inputted into the network along with a selection of desired time points. A sequence of 255 FA images ranging from 0 to 254 seconds post-injection was simulated from the color fundus images (see example time points from this series in FIG. 6). This procedure was performed for several other color fundus images in the test set and found to have satisfactory results, suitable for use as an fluorescein angiogram simulator. Following fluorescein angiogram simulation from a color fundus image, the mean

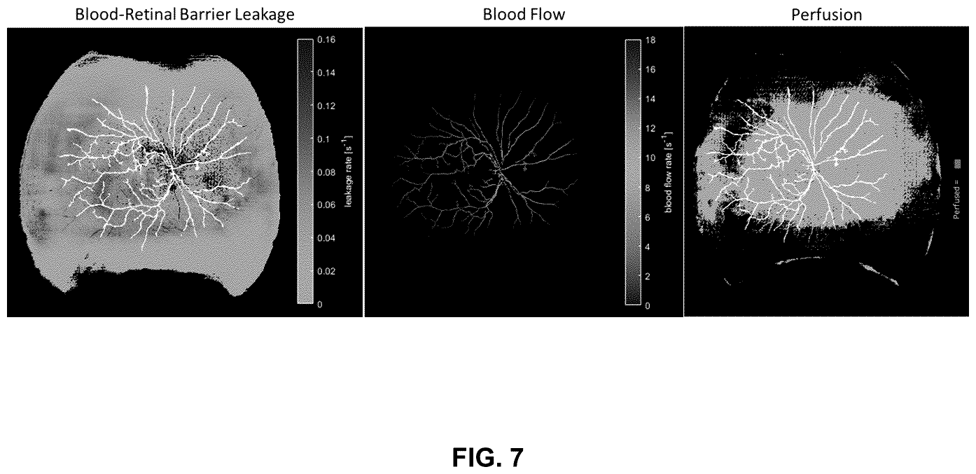

intensity of each simulated FA image was calculated, and the image with maximal fluorescence was identified (hereafter, the “reference image”). The reference image was processed via a blood vessel segmentation algorithm and a binary map of the vasculature was simulated. Each simulated FA image was masked using the binary vasculature map (i.e., pixels outside the vasculature were assigned values of NaN) and the mean-intensity was calculated for all time points. The result is a curve of mean vasculature pixel intensities (FIG. 6). Four time points of interest were selected: choroidal flush, arteriovenous phase, the start of recirculation, and the end of late-phase. Analysis then generally followed the methods described in PCT/CA2022/050926. The rate of early phase intensity change (i.e., blood flow) was computed by performing a pixel-wise linear regression on images between the choroidal flush and the arteriovenous phase. The perfusion map was computed via a binarization of the blood flow map where pixels with a value greater than 0 were assigned a value of 1 (perfused) and pixels with a value equal to or lower than 0 were assigned a value of 0 (non-perfused). The rate of late phase intensity change (i.e., blood-retinal barrier leakage) was computed by performing a pixel-wise linear regression on images between the start of recirculation, and the end of late-phase. Maps were calculated and visualized using colormaps and with manually identified endpoints (FIG. 7). These maps were found to correspond to similarly calculated maps from the real fluorescein angiogram corresponding to this color fundus image.

### Example 2

Conventional GAN Training and RETICAD-Augmented GAN Training: Simulation of a Fluorescein Angiogram from a Color Fundus Image Followed by Analysis of the Simulated Fluorescein Angiogram

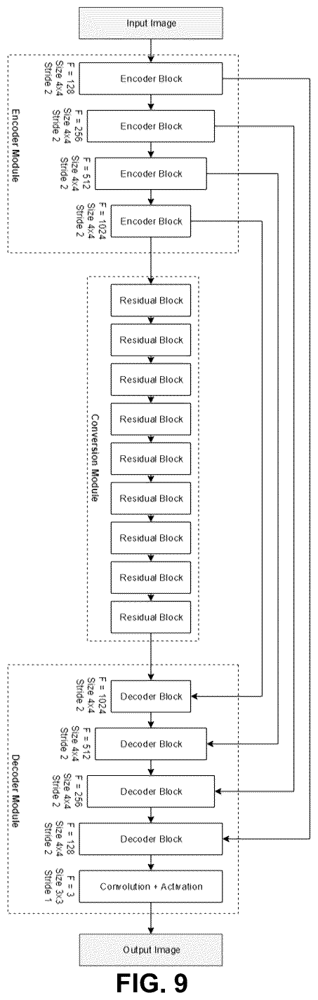

A network was designed (FIG. 9) and trained using two different training methods: conventional GAN training (FIG. 5) and RETICAD-augmented GAN training (FIG. 10). RETICAD-augmented GAN training consisted of 2 additional discriminators, one trained with maps of BRB leakage and the other trained with maps of perfusion.

The training data consisted of 136 FA image sequences (2,818 images) and corresponding color fundus images (136 images).

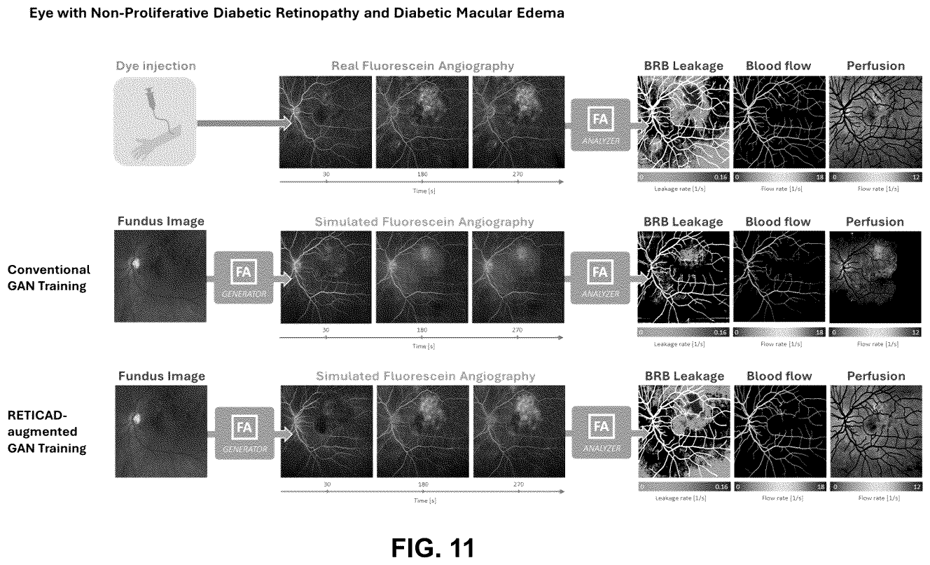

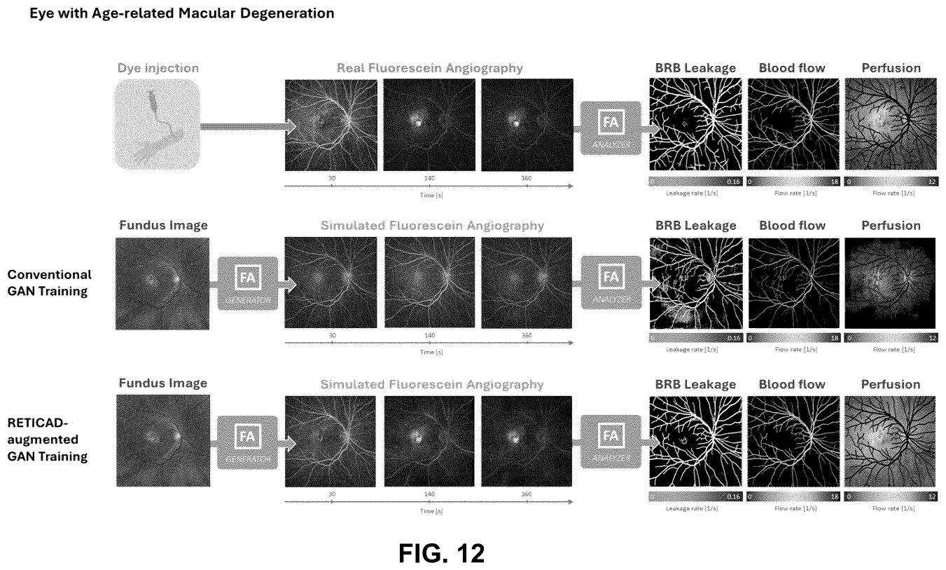

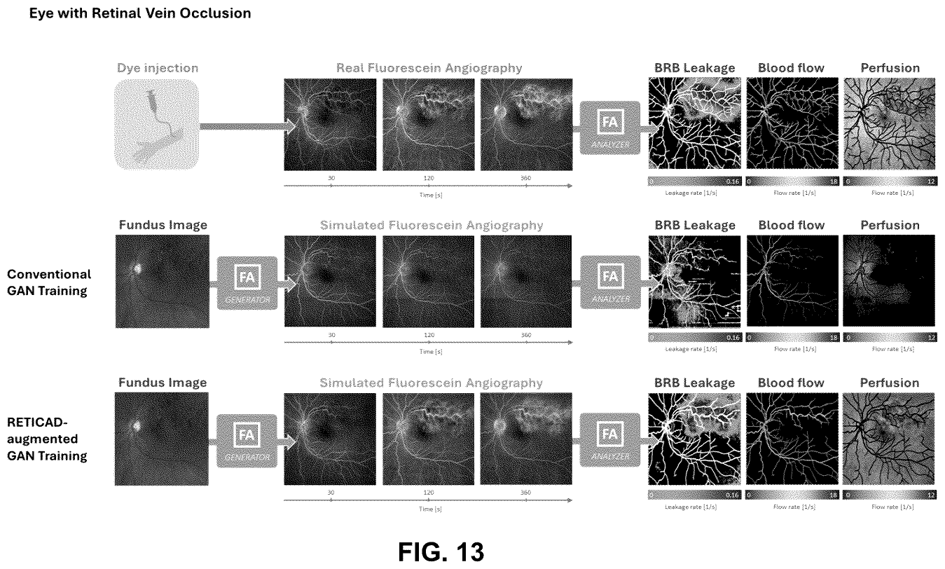

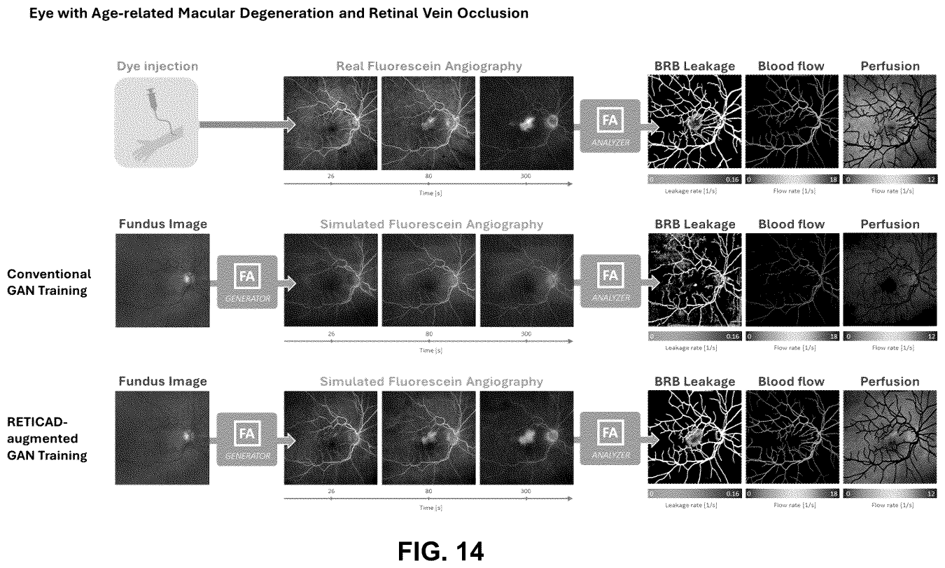

Color fundus images from four eyes were selected from a test set and inputted into the two networks along with the time points of the corresponding real fluorescein angiogram. The four selected eyes are of four patients with the following retinal diseases: non-proliferative diabetic retinopathy and diabetic macular edema (FIG. 11); age-related macular degeneration (FIG. 12); retinal vein occlusion (FIG. 13); and age-related macular degeneration with retinal vein occlusion (FIG. 14). For each patient, the corresponding figure shows three exemplary FA images that are either real, simulated with a network trained using conventional GAN training, or simulated with a network trained using RETICAD-enhanced GAN training. The three types of exemplary FA images are accompanied by the maps calculated from each fluorescein angiogram as described in herein.

Accordingly, the methods disclosed herein have been shown not only to predict or simulate a fluorescein angiogram from a color fundus image, but also to be of sufficient quality to produce analytic output similar to that of an actual FA. Such analytic output is useful for decision support for a known or suspected retinopathy, age-related macular degeneration, or retinal vein occlusion. For illustrative purposes and without limitation, the simulation of FA may also be used in other applications such as neurovascular conditions, cardiovascular conditions, and other direct and indirect vascular indications.

1. A computer implemented method for generating, from a fundus image, a simulated fluorescein angiogram at a set of one or more time points, the method comprising generating simulated fluorescein angiography images for each of the time points using an artificial intelligence (AI) model, the model having been previously trained using previously captured fundus images and associated fluorescein angiograms.

2. The method of claim 1, wherein the generation of simulated fluorescein angiography images comprises:
defining a multi-channel two-dimensional pixel array for each of the set of one or more time points, wherein one or more of the channels of the pixel array comprise pixel values from the fundus image and a third channel of the pixel array comprises encoded time;
generating simulated fluorescein angiography images for each of the time points using the AI model, the AI model taking the multi-channel two-dimensional pixel array as input; and
outputting the simulated fluorescein angiography images.

3. The method of claim 2, wherein the AI model comprises a generator network.

4. The method of claim 3, wherein the fundus image is a color fundus image.

5. The method of claim 4, further comprising determining a mean intensity for each simulated fluorescein angiography image to determine fluorescence, and outputting the simulated fluorescein angiography image with maximal fluorescence.

6. The method of claim 2, wherein the encoded time comprises a time point relative to a choroidal phase, with respect to a maximum possible time for a fluorescein angiography sequence.

7. The method of claim 2, wherein the encoded time is calculated using a linear function or a logarithmic function.

8. The method of claim 3, wherein the generator network comprises a plurality of encoder blocks to compress each color fundus image to a feature representation.

9. The method of claim 8, wherein the generator network further comprises a plurality of residual blocks to convert the feature representation of each color fundus image into a feature representation of the corresponding simulated fluorescein angiography image.

10. The method of claim 10, wherein the generator network further comprises a plurality of decoder blocks to convert the feature representation of the simulated fluorescein angiography image into the simulated fluorescein angiography image.

11. The method of claim 10, wherein the encoder blocks comprise a convolutional layer followed by an activation layer, the residual blocks comprise a convolutional layer followed by an activation layer, and the decoder blocks comprise a transpose convolutional layer followed by an activation layer or an upsampling layer followed by a convolutional layer and an activation layer.

12. The method of claim 11, wherein the residual blocks further comprise a second convolutional layer after the activation layer, wherein the first convolutional layer, the activation layer, and the second convolutional later are concatenated with an input to the respective residual block and passed through a second activation layer.

13. The method of claim 12, wherein the generator network is trained with an image discriminator network, the image discriminator network outputs an array of values delineating regions within the simulated fluorescein angiography image that are determined to be simulated, and wherein a goal of training of the generator network comprises reducing elements within the array of values.

14. The method of claim 13, wherein the generator network is trained using a combination of image training and functional training, the image training comprising using the image discriminator network to reduce elements within the array of values, the functional training comprising a map discriminator network to compare maps of vascular-function determined from a sequence of images from the previously captured fluorescein angiograms and maps of vascular function determined from a sequence of simulated fluorescein angiography images.

15. The method of claim 14, wherein the outputs of the image discriminator network and the map discriminator network are combined to determine a binary cross entropy loss, which is minimized during training of the generator network.

16. The method of claim 14, wherein the maps of vascular function comprise one or more of a retinal perfusion map, a retinal blood flow map, a blood-retinal barrier leakage map, and a leaky and non-leaky microaneurysms map.

17. A system for generating, from a fundus image, a simulated fluorescein angiogram at a set of one or more time points, the system comprising one or more processors and a data memory to execute generating simulated fluorescein angiography images for each of the time points using an artificial intelligence (AI) model, the model having been previously trained using previously captured fundus images and associated fluorescein angiograms.

18. The system of claim 17, wherein the generation of simulated fluorescein angiography images comprises:
defining a multi-channel two-dimensional pixel array for each of the set of one or more time points, wherein one or more of the channels of the pixel array comprise pixel values from the fundus image and a third channel of the pixel array comprises encoded time; and
generating simulated fluorescein angiography images for each of the time points using the AI model, the AI model taking the multi-channel two-dimensional pixel array as input;
and the system further executes outputting the simulated fluorescein angiography images.

19. The system of claim 18, the processors further executing: determining a mean intensity for each simulated fluorescein angiography image to determine fluorescence; and outputting the simulated fluorescein angiography image with maximal fluorescence.

20. A computer implemented method for training an artificial intelligence (AI) model for generating, from a fundus image, a simulated fluorescein angiogram comprising simulated fluorescein angiography images corresponding to a set of one or more time points, wherein the AI model is trained using a combination of image training and functional training, the method comprising:
supplying previously captured fundus images and associated fluorescein angiograms as input to an untrained AI model;
performing image training comprising building an image discriminator network, wherein the image discriminator network outputs an array of values delineating regions within the simulated fluorescein angiography image that are determined to be simulated, and wherein a goal of training of the AI model comprises reducing elements within the array of values; and
performing functional training comprising building a map discriminator network to compare maps of vascular-function determined from the previously captured associated fluorescein angiograms and maps of vascular-function determined from the simulated fluorescein angiography images.
